(19) United States
(12) Patent Application Publication
(10) Pub. No.: US 2026/0347294 A1
(43) Pub. Date: Nov. 5, 2026

(54) METHANOL SYNTHESIS WITH FLUCTUATING RAW GAS SUPPLY

(71) Applicant: L'Air Liquide, Société Anonyme pour I'Etude et I'Exploitation des Procédés Georges Claude, Paris (FR)

(72) Inventors: Vincent PENA, Frankfurt (DE); Sebastian KOENIG, Frankfurt (DE)

(21) Appl. No.: 19/485,969

(22) PCT Filed: May 22, 2024
(86) PCT No.: PCT/EP2024/064139

§ 371 Date: Nov. 19, 2025

(30) Foreign Application Priority Data
Jun. 7, 2023 (EP) 23177898.6

(51) Int. Cl. C07C 29/152; B01J 4/00; B01J 12/00; B01J 19/24

(52) U.S. Cl. CPC C07C 29/152; B01J 4/001; B01J 12/005; B01J 19/24; B01J 2204/002

(57) ABSTRACT

An assembly for methanol synthesis, including a raw gas source, an intermediate storage means, a compressor, and a synthesis reactor assembly. Wherein the raw gas source is connected to the compressor via a first primary conduit. Wherein the compressor is connected to the synthesis reactor assembly via a second primary conduit. Wherein the second primary conduit is connected to an inlet of the intermediate storage means via a first secondary conduit. And wherein an outlet of the intermediate storage means is connected to the second primary conduit via a second secondary conduit and/or connected to the first primary conduit via a third secondary conduit. And a process for operating this assembly.

### CROSS REFERENCE TO RELATED APPLICATIONS

This application is a 371 of International Application No. PCT/EP2024/064139, filed May 22, 2024, which claims priority to European Patent Application No. 23177898.6, filed Jun. 7, 2023, the entire contents of which are incorporated herein by reference.

### BACKGROUND

The present invention relates to an assembly and a process for methanol synthesis with fluctuating raw gas supply.

Conventional plants for methanol production are designed for continuous production at constant loading. Changes in plant loading are minimal in normal operation. This is made possible by continuous supplying with synthesis gases. Loading changes and process adjustments between defined steady states are carried out very slowly over hours to days to protect the methanol plant and the catalyst. Customary synthesis gases from industrial processes contain mainly hydrogen, carbon monoxide and carbon dioxide.

The production of methanol with hydrogen, wherein the hydrogen is produced from renewable energies using an electrolyzer, faces the challenge that hydrogen production is subject to fluctuations. Fluctuations in the amount of hydrogen produced from renewable energies depend on the available solar energy and the available wind energy for example. For example solar energy falls at night or wind power falls during a summer lull. Here, typical loading changes can occur in the space of a few seconds to minutes. Changes to the loading of the electrolyzer are possible in a similar order of magnitude. However, loading changes in the case of fluctuations in renewable energies and of the electrolyzer are an order of magnitude faster than the loading changes of the methanol plant. Furthermore, the permissible operating range of the methanol plant is more limited than that of the electrolyzer and renewable energies.

Methanol synthesis is relatively slow. This makes it the dominant bottleneck, since it is directly connected to the electrolyzer.

Plants with an intermediate storage means for buffering these fluctuations are known in the literature. In such a plant the intermediate storage means is placed between the electrolyzer and the methanol plant.

However, the known prior art has the disadvantage that if no intermediate storage means is employed excess hydrogen resulting from an overcapacity of hydrogen must be disposed of. This may even result from the mere fact that the methanol plant is operated under partial loading and the electrolyzer spontaneously provides more hydrogen again. On the other hand, in the event of a rapid fall in the hydrogen amount, the methanol plant must temporarily be operated with conventional hydrogen from fossil sources or the methanol plant must be shut down for these cases.

### SUMMARY

Proceeding therefrom it is an object of the present invention to integrate an intermediate storage means as efficiently as possible into the overall process of a methanol synthesis and to enable reliable operation of the methanol synthesis in the case of a fluctuating raw gas source.

This object is achieved with the assembly and the process according to the independent claims. Further advantageous embodiments of the invention are specified in the dependent claims. The features individually listed in the dependent claims may be combined with one another in technologically advantageous fashion and may define further embodiments of the invention. Moreover, the features specified in the claims are more particularly described and elucidated in the description, with further preferred embodiments of the invention being specified.

The present invention provides an assembly for methanol synthesis which comprises a raw gas source and an intermediate storage means and a first compressor and a synthesis reactor assembly. The raw gas source is connected to the first compressor via a first primary conduit. The first compressor is connected to the synthesis reactor assembly via a second primary conduit. The second primary conduit is connected to an inlet of the intermediate storage means via a first secondary conduit. An outlet of the intermediate storage means is connected to the second primary conduit via a second secondary conduit and/or connected to the first primary conduit via a third secondary conduit.

The raw gas source is adapted to provide one or more components of a raw gas. Accordingly the raw gas source is adapted to provide one or more raw gas streams. It is immaterial to the mode of operation of the described assembly whether the raw gas used for the methanol synthesis derives from a single raw gas source or from a plurality of separate raw gas sources.

The raw gas stream and/or the raw gas streams can fluctuate. The term “fluctuation” may refer to pressure as well as mass flow and is to be understood as meaning change in pressure or mass flow over time. The raw gas comprises one or more of the reactants of the methanol synthesis. The raw gas may comprise at least one of the gaseous reactants hydrogen, carbon dioxide and/or carbon monoxide. The individual reactants may be provided as a common raw gas stream or as individual raw gas streams. Individual raw gas streams may have different purities. The raw gas may contain concomitants that are inert under the conditions of methanol synthesis, for example nitrogen or methane.

At least one of the raw gas streams preferably derives from a water electrolysis process. The water electrolysis process is preferably operated with a supply of renewable energy which may fluctuate. At least one of the raw gas streams may derive from a carbon dioxide separation process or a carbon dioxide conduit. The raw gas pressure may especially be between 1 bar and 100 bar. The raw gas pressure may be at least 1 bar, preferably at least 30 bar.

The intermediate storage means is used to intermediately store at least one raw gas. This may be effected as required. It is immaterial to the mode of operation of the described assembly whether the raw gas intermediately stored in the intermediate storage means comprises all reactants of the methanol synthesis or only one of these. If the raw gas does not contain all reactants the remaining reactants may be otherwise supplied to the synthesis reactor assembly. The way in which this is done is immaterial to the mode of operation of the described assembly.

The intermediate storage means may be composed of one or more tanks and/or a pipe system and/or one or more caverns. The intermediate storage means may store a maximum net consumption of the raw gas in the methanol synthesis for one minute, preferably for six hours, particularly preferably for more than 12 hours. The pressure in the intermediate storage means is preferably in a range from 5 bar to 700 bar. The pressure is preferably 5 bar to 100 bar, 20 bar to 100 bar or 80 bar to 700 bar. The pressure in the intermediate storage means may be kept constant by adapting the storage volume by connecting or disconnecting tanks, pipe systems and/or caverns. In the case of exclusive supply of the methanol synthesis from the intermediate storage means the pressure in the intermediate storage means may fall below the standard pressure of the raw gas. The temperature of the intermediate storage means is preferably between- 50 °C and 100 °C.

The synthesis reactor assembly is used for synthesizing methanol from the raw gas. A loading of the synthesis reactor assembly, the so-called loading range, is preferably between 10% and 100% at any time of operation of the assembly, preferably between 20% and 100%. A loading range of 100% is also referred to as full loading. A loading range below 100% is also referred to as partial loading. The synthesis reactor assembly may be configured for operation with a minimum partial loading of 20% for example. The pressure in the synthesis reactor assembly is preferably between 20 bar and 200 bar, preferably between 30 bar and 100 bar. The operating temperature of the synthesis reactor assembly is preferably between 150 °C and 300 °C, preferably between 200 °C and 300 °C.

The assembly is used for producing methanol and integrating the intermediate storage means into the overall process as efficiently as possible. The assembly is further used for reliable operation of a synthesis reactor assembly with a fluctuating raw gas supply. This is achieved by the subsequently described incorporation of the intermediate storage means into the assembly.

The raw gas source is connected to the first compressor via a first primary conduit and the first compressor is connected to the synthesis reactor assembly via a second primary conduit. The raw gas source is thus connected to the synthesis reactor assembly via the first compressor. This makes it possible to supply the raw gas to the synthesis reactor assembly. The section of the conduit between the raw gas source and the first compressor is referred to as the first primary conduit and the section of the conduit between the first compressor and the synthesis reactor assembly is referred to as the second primary conduit. The first primary conduit and the second primary conduit may be realized as a single conduit into which the first compressor is integrated.

The second primary conduit is connected to an inlet of the intermediate storage means via a first secondary conduit. Raw gas may also be diverted from the conduit between the raw gas source and the synthesis reactor assembly at a point between the first compressor and the synthesis reactor assembly. Diversion downstream of the first compressor has the advantage that the first compressor may be utilized to compress the raw gas for depositing in the intermediate storage means. Thus, the first compressor is not only used to compress the raw gas in the course of supplying it to the synthesis reactor assembly. A separate compressor for depositing purposes is thus not required.

An outlet of the intermediate storage means is connected to the second primary conduit via a second secondary conduit and/or connected to the first primary conduit via a third secondary conduit. The “and” case is preferred. The raw gas stored in the intermediate storage means can thus be reintroduced into the conduit between the raw gas source and the synthesis reactor assembly downstream of the first compressor and/or upstream of the first compressor. The raw gas is preferably introduced from the intermediate storage means downstream of the compressor, i.e. into the second primary conduit, if the pressure in the intermediate storage means is greater than a threshold value. The threshold value is preferably selected based on the operating pressure of the synthesis reactor assembly. If the pressure in the intermediate storage means is greater than the threshold value a further compression is not required. The raw gas is preferably introduced from the intermediate storage means upstream of the first compressor, i.e. into the first primary conduit, if the pressure in the intermediate storage means is lower than the threshold value. In this case the raw gas withdrawn from the intermediate storage means may be further compressed with the first compressor.

The described configuration has the result that there is no clear division in the described assembly between the source and storage system on the one hand and the synthesis system on the other hand. In a solution with such a separation, as is known from the prior art, the source and storage system would pass a constant raw gas stream to the synthesis system. Fluctuations of the source would be equalized by a storage means within the source and storage system. The synthesis system comprising the compressor would process the constant raw gas stream. In the described assembly the intermediate storage means is connected to the conduit between the raw gas source and the synthesis reactor assembly downstream of the first compressor, i.e. to the second primary conduit. This especially breaks through a separation between the source and storage system and the synthesis system. This allows the above-described utilization of the compressor, which in the described assembly corresponds to a synthesis system, for the purposes of depositing in the intermediate storage means. This compressor is presently referred to as the first compressor.

The raw gas may be deposited in the intermediate storage means as desired and at a later point in time reintroduced from the intermediate storage means into the synthesis reactor assembly via the second primary conduit or via the first primary conduit and the second primary conduit. This may be controlled via pressure regulating devices, in particular via valves. These may be arranged in a very wide variety of ways. Preferred embodiments in this respect will now be described below.

The assembly preferably comprises a first valve between the second primary conduit and the first secondary conduit. The first valve is preferably configured as a 3-way valve which is integrated into the second primary conduit and to which the first secondary conduit is connected as a branch.

The first valve can prevent unwanted pressure equalizations and also reduce dead times. Dead times can occur when the raw gas traverses the first secondary conduit to the intermediate storage means.

The assembly preferably comprises a second valve between the outlet of the intermediate storage means and the second secondary conduit and/or between the outlet of the intermediate storage means and the third secondary conduit. For example the second valve may be configured as a 3-way valve, wherein an inlet of the second valve is connected to the intermediate storage means, wherein the second secondary conduit is connected to a first outlet of the second valve and the third secondary conduit is connected to a second outlet of the second valve. In this case the second secondary conduit and the third secondary conduit are connected to the outlet of the intermediate storage means via a common conduit between the second valve.

The second valve can selectively establish a connection between the outlet of the intermediate storage means and the first primary conduit via the third secondary conduit or between the outlet of the intermediate storage means and the second primary conduit via the second secondary conduit. A second valve can reduce dead times. Dead times can occur when the raw gas from the intermediate storage means traverses the second or third secondary conduit.

The second secondary conduit is preferably connected to the second primary conduit upstream of the connection between the first secondary conduit and the second primary conduit.

This assembly allows recirculation to the intermediate storage means of raw gas which was not able to be converted in the synthesis reactor assembly in the present loading range.

However, the essential mode of operation of the described assembly is also achievable when the second secondary conduit is connected to the second primary conduit downstream of the connection between the first secondary conduit and the second primary conduit.

In a preferred embodiment the first primary conduit is connected to the inlet of the intermediate storage means via a fourth secondary conduit.

In this embodiment raw gas which was not able to be converted in the synthesis reactor assembly in the present loading range may be deposited in the intermediate storage means directly from the first primary conduit.

It is preferable when a third valve connects the fourth secondary conduit with the first primary conduit. The third valve is preferably configured as a 3-way valve which is integrated into the first primary conduit and to which the fourth secondary conduit is connected as a branch. The third valve can divert raw gas at elevated pressure at least partially even upstream of the first compressor and save further compression work in the first compressor.

In a further preferred embodiment the assembly further comprises a second compressor connected in parallel with the first compressor.

In this embodiment the first compressor may be used for depositing raw gas. In particular a component of the raw gas, preferably hydrogen, may be deposited using the first compressor. A further component of the raw gas may be compressed to a suitable operating pressure for the synthesis reactor assembly using the second compressor. A parallel second compressor makes it possible to achieve more flexible operation as well as operation with different raw gas sources.

In a further preferred embodiment a third compressor is connected upstream of the inlet of the intermediate storage means.

In this embodiment the raw gas may additionally be deposited in the intermediate storage means at a higher pressure. The higher pressure makes it possible to deposit more raw gas in the intermediate storage means.

This embodiment no longer achieves the above-described advantage that a separate compressor for depositing in the intermediate storage means can be eschewed. However, compared to solutions from the prior art the requirements of the third compressor of the present embodiment are lower than those of a separate compressor for a source and storage system that is completely separate from the synthesis system.

The third compressor is preferably connected in parallel with a conduit section of the first secondary conduit. If the assembly also comprises the fourth secondary conduit the third compressor is preferably connected in parallel with a conduit section of the first secondary conduit and/or in parallel with a conduit section of the fourth secondary conduit. The “and” case is preferred. This case can be realized when the first secondary conduit and the fourth secondary conduit have a common conduit section. The third compressor is preferably connected in parallel with this common conduit section.

In a further preferred embodiment a turbine is connected downstream of the outlet of the intermediate storage means.

In this embodiment the applied compression energy may be partially converted into mechanical and/or electrical energy. This can increase the overall energy efficiency of the assembly.

If the assembly comprises the second secondary conduit the turbine is preferably connected in parallel with a conduit section of the second secondary conduit. If the assembly comprises the third secondary conduit the turbine is preferably connected in parallel with a conduit section of the third secondary conduit. If the assembly comprises the second secondary conduit and the third secondary conduit the turbine is preferably connected in parallel with a conduit section of the second secondary conduit and/or in parallel with a conduit section of the third secondary conduit. The “and” case is preferred. This case can be realized when the second secondary conduit and the third secondary conduit have a common conduit section. The turbine is preferably connected in parallel with this common conduit section.

In a further preferred embodiment the raw gas source comprises an electrolyzer. In this case it is preferable when the assembly additionally comprises a further raw gas source for carbon dioxide.

In a further preferred embodiment the raw gas source comprises an electrolyzer and a further raw gas source comprises a carbon dioxide conduit or a carbon dioxide separation unit.

In this embodiment methanol produced may be described as at least partially sustainable and/or at least partially renewable. Furthermore, the overall energy efficiency of the methanol synthesis can be increased.

In a further preferred embodiment the raw gas source comprises a first component and a second component. The first component preferably comprises hydrogen (H₂). The second component preferably comprises carbon dioxide.

A further aspect of the invention provides a process for operating an assembly configured as described,
wherein in a first operating mode the raw gas source provides more raw gas than is converted by the synthesis reactor assembly, wherein excess raw gas from the second primary conduit is supplied to the intermediate storage means via the first secondary conduit and deposited in the intermediate storage means,
wherein in a second operating mode the raw gas source provides less raw gas than is converted by the synthesis reactor assembly and the raw gas in the intermediate storage means is at a pressure above a threshold value, wherein raw gas from the intermediate storage means is introduced into the second primary conduit via the second secondary conduit and
wherein in a third operating mode the raw gas source provides less raw gas than is converted by the synthesis reactor assembly and the raw gas in the intermediate storage means is at a pressure below the threshold value, wherein the raw gas from the intermediate storage means is introduced into the first primary conduit via the third secondary conduit.

The advantages and features of the assembly for methanol synthesis are applicable and transferable to the process, and vice versa. The assembly is preferably adapted for operation according to the described process.

The assembly will operate in the operating mode suitable in each case depending on the circumstances. It is not necessary to run through all three described operating modes. There is also no fixed sequence of the operating modes. On the contrary it is preferable to monitor how much raw gas is provided by the raw gas source and the state of the intermediate storage means and to decide the operating mode in which to operate the assembly on the basis of the results of this monitoring. There may be further operating modes in addition to the described three operating modes.

The first operating mode is utilized when the raw gas source provides more raw gas than is converted by the synthesis reactor assembly. The condition that more raw gas is present than can be converted by the synthesis reactor assembly is based on different scenarios. In a first scenario the synthesis reactor assembly is operated at full loading. The raw gas source spontaneously produces more raw gas than can be converted in the synthesis reactor assembly.

In a second scenario the synthesis reactor assembly is operated under partial loading. The raw gas source spontaneously produces more raw gas than can be converted in this partial loading condition. The synthesis reactor assembly cannot follow the increase in raw gas amount on account of its slow response.

In both scenarios the excess amount of raw gas would be ejected as an offgas upstream and/or downstream of the synthesis reactor assembly without depositing in the intermediate storage means.

By contrast, the first operating mode effects resource-saving depositing of the raw gas.

In the second operating mode and in the third operating mode the raw gas source provides less raw gas than is converted by the synthesis reactor assembly. The condition that the raw gas source provides less raw gas than the synthesis reactor assembly can convert is likewise based on different scenarios.

In a third scenario the synthesis reactor assembly is operated at full loading. The raw gas source spontaneously produces less raw gas than can be converted in the full loading condition. The synthesis reactor assembly cannot sufficiently follow the decrease in raw gas amount on account of its slow response.

In a fourth scenario the synthesis reactor assembly is operated at partial loading. The raw gas source spontaneously produces less raw gas than can be converted in the partial loading state or none at all. The synthesis reactor assembly cannot sufficiently follow the decrease in raw gas amount on account of its slow response. The synthesis reactor assembly may further have a minimum partial loading below which the synthesis reactor assembly is shut down.

Without introduction of the raw gas from the intermediate storage means the third and fourth scenario can result in introduction of a fossil raw gas to compensate the missing raw gas or a costly and complex shutdown of the synthesis reactor assembly. In the second operating mode and in the third operating mode this may be prevented by introducing raw gas from the intermediate storage means.

In the second operating mode the raw gas in the intermediate storage means is at a pressure above a threshold value, wherein raw gas from the intermediate storage means is introduced into the second primary conduit via the second secondary conduit.

If the raw gas in the intermediate storage means is at a pressure above a threshold value the raw gas may be introduced without further compression.

The threshold value can follow from the operating pressure of the synthesis reactor assembly and the compensation of possible pressure losses between the intermediate storage means and the synthesis reactor assembly.

The second operating mode is used for energy-saving introduction of raw gas for reliable operation of the synthesis reactor assembly.

In the third operating mode the raw gas in the intermediate storage means is at a pressure below the threshold value, wherein the raw gas from the intermediate storage means is introduced into the first primary conduit via the third secondary conduit.

In the third operating mode the raw gas from the intermediate storage means may be compressed to above the threshold value with the first compressor.

The third operating mode is used for energy-saving introduction of raw gas and more efficient utilization of the storage volume of the intermediate storage means for reliable operation of the synthesis reactor assembly.

For reaction in the synthesis reactor assembly in the second and in the third operating mode the missing raw gas which is not sufficiently provided by the raw gas source can be withdrawn from the intermediate storage means and, depending on the pressure in the intermediate storage means, introduced into the first primary conduit or into the second primary conduit.

If the assembly comprises the fourth secondary conduit the assembly may also be operated in a fourth operating mode. In this mode the raw gas source provides more raw gas than is converted by the synthesis reactor assembly, wherein excess raw gas from the first primary conduit is supplied to the intermediate storage means via the fourth secondary conduit and deposited in the intermediate storage means.

In a preferred embodiment of the process the raw gas from the raw gas source comprises hydrogen which is at least partially obtained by electrolysis of water with renewable energy.

A first component of the raw gas can be compressed in the first compressor and a second component of the raw gas can be compressed in the parallel second compressor provided the assembly comprises such a compressor. The first component and the second component of the raw gas may subsequently be mixed.

The first compressor may be used for depositing raw gas. In particular a first component of the raw gas, preferably hydrogen, may be deposited using the first compressor. A second component of the raw gas may be compressed to a suitable operating pressure for the synthesis reactor assembly using the second compressor. A parallel second compressor can allow more flexible operation.

If the assembly comprises a third compressor in the first operating mode said compressor can supply the raw gas from the first secondary conduit and/or the fourth secondary conduit to the intermediate storage means. The third compressor can deposit the raw gas in the intermediate storage means at a higher pressure. The higher pressure makes it possible to deposit more raw gas in the intermediate storage means.

If the assembly comprises a turbine in the second operating mode and/or in the third operating mode said turbine can convert decompression work into electrical and/or mechanical work. The turbine can increase overall energy efficiency.

Heat generated in one of the compressors may be utilized for heating consumers in the raw gas production and/or the synthesis and/or to provide thermal energy to an external consumer. This can increase overall energy efficiency.

### BRIEF DESCRIPTION OF THE FIGURES

The invention will be explained in more detail below with reference to the figures. The figures show particularly preferred exemplary embodiments but the invention is not limited to these. The figures and the size relationships presented therein are only schematic. In the figures:

FIG. 1 is an assembly known from the prior art for methanol synthesis in a schematic representation,

FIG. 2 is a schematic representation of a first embodiment of an inventive assembly for methanol synthesis,

FIG. 3 is a schematic representation of a second embodiment of an inventive assembly for methanol synthesis,

FIG. 4 is a schematic representation of a third embodiment of an inventive assembly for methanol synthesis,

FIG. 5 is a schematic representation of a fourth embodiment of an inventive assembly for methanol synthesis.

### DETAILED DESCRIPTION OF THE PREFERRED EMBODIMENTS

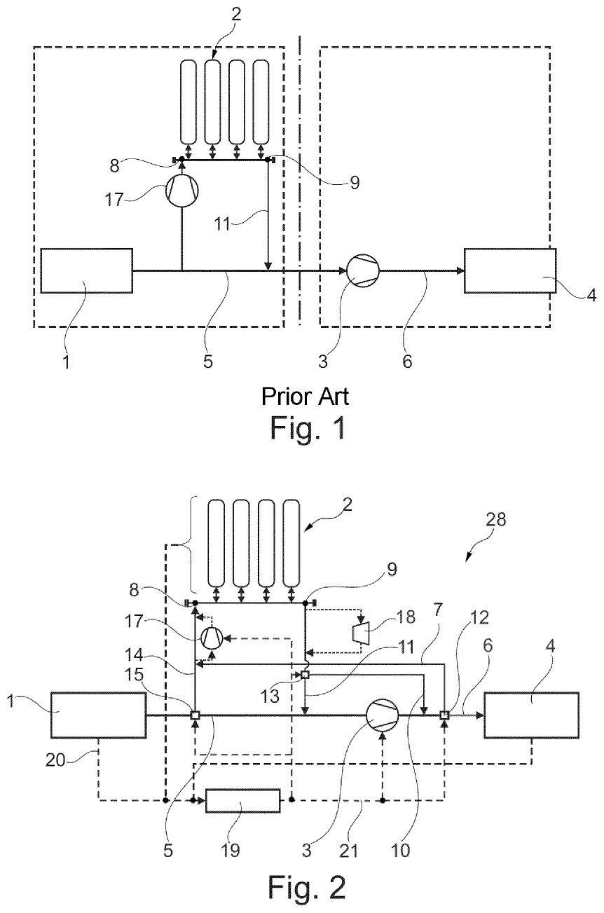

FIG. 1 shows an assembly known from the prior art for methanol synthesis. The assembly comprises a raw gas source 1, a first primary conduit 5, a second primary conduit 6, a first compressor 3, a third compressor 17, a synthesis reactor assembly 4, an intermediate storage means 2 and a third secondary conduit 11. The raw gas source 1 is connected to the first compressor 3 via the first primary conduit 5. The first compressor 3 is connected to the synthesis reactor assembly 4 via the second primary conduit 6. An inlet 8 of the intermediate storage means 2 is connected to the first primary conduit 5 via the third compressor 17. The third secondary conduit 11 connects an outlet 9 of the intermediate storage means 2 to the first primary conduit 5. The raw gas is diverted from the primary conduit 5 and deposited in the intermediate storage means 2 using the third compressor 17. If necessary the raw gas from the intermediate storage means 2 is compressed to an operating pressure of the synthesis reactor assembly 4 using the first compressor 3 via the third secondary conduit 11. A dashed line indicates that the assembly known from the prior art is divided into a source and storage system (left-hand side) and a synthesis system (right-hand side). This entails that during depositing in the intermediate storage means 2 the raw gas must be compressed by the third compressor 17 alone. Said compressor must be configured accordingly which is disadvantageous.

FIG. 2 shows a schematic representation of an inventive assembly 28 for methanol synthesis. The assembly 28 comprises a raw gas source 1, an intermediate storage means 2, a compressor 3 and a synthesis reactor assembly 4. The raw gas source 1 comprises an electrolyzer. The raw gas from the raw gas source 1 comprises hydrogen which is partially obtained by electrolysis of water with renewable energy. The raw gas further comprises carbon dioxide and/or carbon monoxide. The intermediate storage means 2 consists of a plurality of tanks and a pipe system. The raw gas source 1 is connected to the compressor 3 via a first primary conduit 5, wherein the compressor 3 is connected to the synthesis reactor assembly 4 via a second primary conduit 6. The second primary conduit 6 is connected to an inlet 8 of the intermediate storage means 2 via a first secondary conduit 7. An outlet 9 of the intermediate storage means 2 is connected to the second primary conduit 6 via a second secondary conduit 10 and connected to the first primary conduit 5 via a third secondary conduit 11. A first valve 12 is arranged between the second primary conduit 6 and the first secondary conduit 7. A second valve 13 is arranged between the outlet 9 of the intermediate storage means 2 and the second secondary conduit 10 and the third secondary conduit 11. The second secondary conduit 10 is connected upstream of the connection between the first secondary conduit 7 and the second primary conduit 6 to the second primary conduit 6. The first primary conduit 5 is connected to the inlet 8 of the intermediate storage means 2 via a fourth secondary conduit 14. A third valve 15 is arranged between the fourth secondary conduit 14 and the first primary conduit 5. A third compressor 17 is connected upstream of the inlet 8 of the intermediate storage means 2. A turbine 18 is connected downstream of the outlet 9 of the intermediate storage means 2. A control unit 19 is provided for controlling the valves 12;13;15 and the compressors 3;17.

The assembly 28 may be operated for methanol synthesis in the following three operating modes.

In a first operating mode the raw gas source 1 provides more raw gas than is converted by the synthesis reactor assembly 4, wherein excess raw gas from the second primary conduit 6 is supplied to the intermediate storage means 2 via the first secondary conduit 7 and deposited in the intermediate storage means 2. The raw gas can be compressed via the third compressor 17.

In a second operating mode the raw gas source 1 provides less raw gas than is converted by the synthesis reactor assembly 4. Furthermore, in the second operating mode the raw gas in the intermediate storage means 2 is at a pressure above a threshold value, wherein raw gas from the intermediate storage means 2 is introduced into the second primary conduit 6 via the second secondary conduit 10.

In a third operating mode the raw gas source 1 provides less raw gas than is converted by the synthesis reactor assembly 4. In the third operating mode the raw gas in the intermediate storage means 2 is at a pressure below the threshold value, wherein the raw gas from the intermediate storage means 2 is introduced into the first primary conduit 5 via the third secondary conduit 11.

In the second and third operating mode the decompression work of the turbine 18 may be converted into electrical power.

In a fourth operating mode the raw gas source 1 provides more raw gas than is converted by the synthesis reactor assembly 4, wherein excess raw gas from the first primary conduit 5 is supplied to the intermediate storage means 2 via the fourth secondary conduit 14 and deposited in the intermediate storage means 2. The raw gas can be compressed via the third compressor 17.

The control unit 19 controls the valves 12;13;15 and the compressors 3;17 according to the first, second or third operating mode. The control unit 19 can receive input parameters 20 from the raw gas source 1 about the amount of the produced raw gas, from the intermediate storage means 2 about the pressure and the fill level and from the synthesis reactor assembly 4 about the loading. The control unit 19 controls the valves 12;13;15 and the compressors 3;17 via output parameters 21.

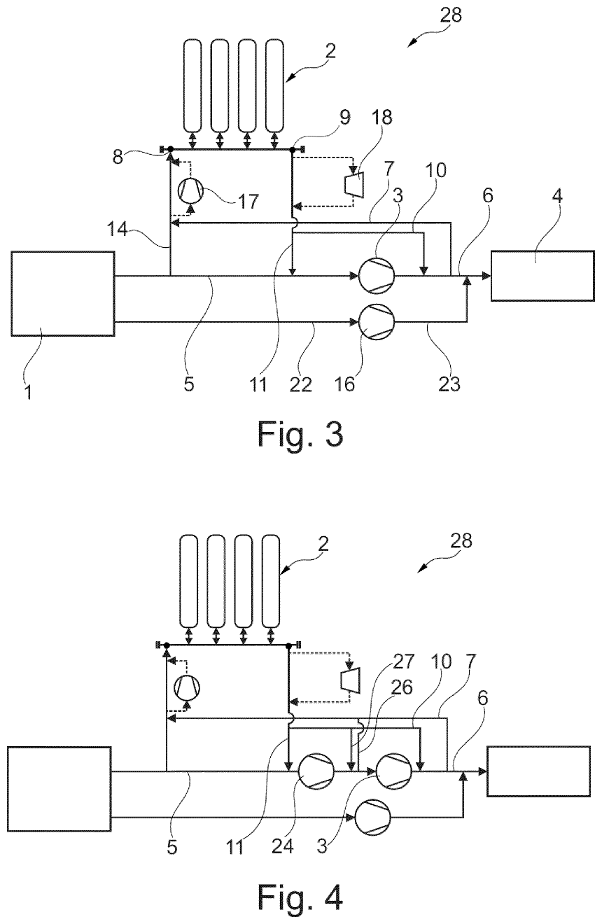

FIG. 3 shows a schematic representation of a second embodiment of an inventive assembly 28 for methanol synthesis. In addition to the components shown in FIG. 2 the assembly 28 comprises a second compressor 16 connected in parallel with the first compressor 3. The first primary conduit 5 and the second primary conduit 6 are used for supplying a first component of the raw gas to the synthesis reactor assembly 4. The first component of the raw gas from the raw gas source 1 is compressed in the first compressor 3.

The raw gas source 1 is connected to the second compressor 16 via a third primary conduit 22. The second compressor 16 is connected to the synthesis reactor assembly 4 via a fourth primary conduit 23. The third primary conduit 22 and the fourth primary conduit 23 are used for supplying a second component of the raw gas to the synthesis reactor assembly 4. The second component of the raw gas from the raw gas source 1 is compressed in the second compressor 16. The second component of the raw gas is introduced into the second primary conduit 6 via the fourth primary conduit 23.

The first compressor 3 is used for depositing the first component of raw gas in the intermediate storage means 2. The second component of the raw gas is compressed to a suitable operating pressure for the synthesis reactor assembly 4 using the second compressor 16. The parallel second compressor 16 allows more flexible operation.

FIG. 4 shows a schematic representation of a third embodiment of an inventive assembly 28 for methanol synthesis. In addition to the elements shown in FIG. 3 the assembly 28 from FIG. 4 further comprises a fourth compressor 24 arranged between the introduction of the third secondary conduit 11 into the first primary conduit 5 and the first compressor 3. Between the fourth compressor 24 and the first compressor 3 a fifth secondary conduit 26 diverts raw gas to the first secondary conduit 7 and a sixth secondary conduit 27 introduces raw gas from the secondary conduit 10.

In this embodiment the raw gas may be withdrawn from the intermediate storage means 2 at different pressure ranges and introduced into the first and second primary conduit 5;6. This allows for more flexible operation.

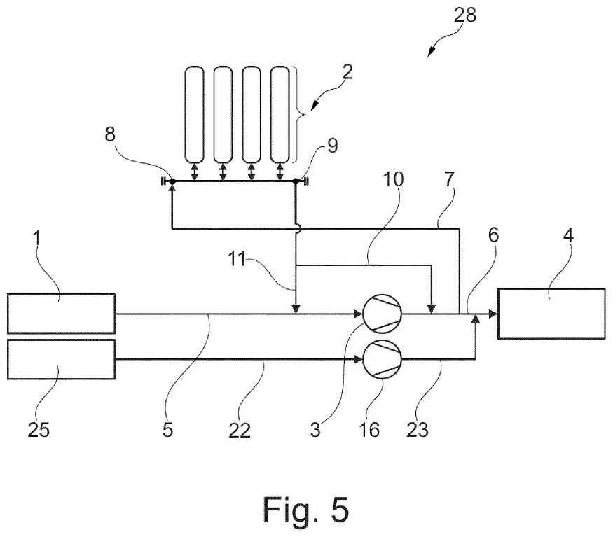

FIG. 5 shows a schematic representation of a fourth embodiment of an inventive assembly 28 for methanol synthesis. The assembly 28 comprises a raw gas source 1, a further raw gas source 25, an intermediate storage means 2, a first compressor 3, a second compressor 16 and a synthesis reactor assembly 4. The raw gas source 1 for a first component of the raw gas is an electrolyzer. The raw gas from the raw gas source 1 is hydrogen which is partially obtained by electrolysis of water with renewable energy. The further raw gas source 25 for a second component of the raw gas produces carbon dioxide from a carbon dioxide separation process. The carbon dioxide separation process of the further raw gas source 25 operates constantly and does not require intermediate storage in the intermediate storage means 2.

The intermediate storage means 2 consists of a plurality of tanks and a pipe system. The raw gas source 1 is connected to the compressor 3 via a first primary conduit 5, wherein the compressor 3 is connected to the synthesis reactor assembly 4 via a second primary conduit 6.

The further raw gas source 25 is connected to the second compressor 16 via a third primary conduit 22. The second compressor 16 is connected to the second primary conduit 6 via a fourth primary conduit 23. The second compressor 16 is connected to the synthesis reactor assembly 4 via the fourth primary conduit 23.

The second primary conduit 6 is connected to an inlet 8 of the intermediate storage means 2 via a first secondary conduit 7. An outlet 9 of the intermediate storage means 2 is connected to the second primary conduit 6 via a second secondary conduit 10 and connected to the first primary conduit 5 via a third secondary conduit 11. The second secondary conduit 10 is connected upstream of the connection between the first secondary conduit 7 and the second primary conduit 6 to the second primary conduit 6. The assembly 28 may be operated for methanol synthesis in the following three operating modes.

In a first operating mode the raw gas source 1 provides more raw gas than is converted by the synthesis reactor assembly 4, wherein excess raw gas from the second primary conduit 6 is supplied to the intermediate storage means 2 via the first secondary conduit 7 and deposited in the intermediate storage means 2.

In a second operating mode the raw gas source 1 provides less raw gas than is converted by the synthesis reactor assembly 4. Furthermore, in the second operating mode the raw gas in the intermediate storage means 2 is at a pressure above a threshold value, wherein raw gas from the intermediate storage means 2 is introduced into the second primary conduit 6 via the second secondary conduit 10.

In a third operating mode the raw gas source 1 provides less raw gas than is converted by the synthesis reactor assembly 4. In the third operating mode the raw gas in the intermediate storage means 2 is at a pressure below the threshold value, wherein the raw gas from the intermediate storage means 2 is introduced into the first primary conduit 5 via the third secondary conduit 11.

During the first, second and third operating mode the raw gas source 25 supplies the second component of the raw gas constantly.

### REFERENCE NUMERALS

1 Raw gas source
2 Intermediate storage means
3 First compressor
4 Synthesis reactor assembly
5 First primary conduit
6 Second primary conduit
7 First secondary conduit
8 Inlet
9 Outlet
10 Second secondary conduit
11 Third secondary conduit
12 First valve
13 Second valve
14 Fourth secondary conduit
15 Third conduit
16 Second compressor
17 Third compressor
18 Turbine
19 Control unit
20 Input parameters
21 Output parameters
22 Third primary conduit
23 Fourth primary conduit
24 Fourth compressor
25 Second raw gas source
26 Fifth secondary conduit
27 Sixth secondary conduit
28 Assembly

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

9. An assembly for methanol synthesis, comprising:
a raw gas source,
an intermediate storage means,
a compressor, and
a synthesis reactor assembly,
wherein the raw gas source is connected to the compressor via a first primary conduit, wherein the compressor is connected to the synthesis reactor assembly via a second primary conduit,
wherein the second primary conduit is connected to an inlet of the intermediate storage means via a first secondary conduit,
wherein an outlet of the intermediate storage means is connected to the second primary conduit via a second secondary conduit and/or connected to the first primary conduit via a third secondary conduit.

10. The assembly according to claim 9, wherein the first primary conduit is connected to the inlet of the intermediate storage means via a fourth secondary conduit.

11. The assembly according to claim 9, further comprising a second compressor connected in parallel with the first compressor.

12. The assembly according to claim 9, wherein a third compressor is connected upstream of the inlet of the intermediate storage means.

13. The assembly according to claim 9, wherein a turbine is connected downstream of the outlet of the intermediate storage means.

14. The assembly according to claim 9, wherein the raw gas source comprises an electrolyzer.

15. A process for operating an assembly according to claim 9,
wherein in a first operating mode the raw gas source provides more raw gas than is converted by the synthesis reactor assembly, wherein excess raw gas from the second primary conduit is supplied to the intermediate storage means via the first secondary conduit and deposited in the intermediate storage means,
wherein in a second operating mode the raw gas source provides less raw gas than is converted by the synthesis reactor assembly and the raw gas in the intermediate storage means is at a pressure above a threshold value, wherein raw gas from the intermediate storage means is introduced into the second primary conduit via the second secondary conduit, and
wherein in a third operating mode the raw gas source provides less raw gas than is converted by the synthesis reactor assembly and the raw gas in the intermediate storage means is at a pressure below the threshold value, wherein the raw gas from the intermediate storage means is introduced into the first primary conduit via the third secondary conduit.

16. The process according to claim 15, wherein the raw gas from the raw gas source) comprises hydrogen which is at least partially obtained by electrolysis of water with renewable energy.
